(19) United States
(12) Patent Application Publication
(10) Pub. No.: US 2025/0333784 A1
(43) Pub. Date: Oct. 30, 2025

(54) Systems and Methods for Sequencing Dissociation Spectra of Oligonucleotides Obtained by Negative Electron Activated Dissociation

(71) Applicant: DH Technologies Development Pte. Ltd., Singapore (SG)

(72) Inventors: Takashi BABA, Richmond Hill (CA); Kaoru KARASAWA, Tokyo (JP)

(21) Appl. No.: 18/870,107

(22) PCT Filed: May 26, 2023
(86) PCT No.: PCT/IB2023/055445

§ 371 Date: Nov. 27, 2024

Related U.S. Application Data
(60) Provisional application No. 63/347,946, filed on Jun. 1, 2022.

(51) Int. Cl. C12Q 1/6872; H01J 49/00

(52) U.S. Cl. CPC C12Q 1/6872; H01J 49/0036

(57) ABSTRACT

A system for analyzing mass spectra of a deprotonated oligonucleotide comprises a mass spectrometer configured to collect mass spectrometry data and an analyzer module configured to receive and analyze the mass spectrometry data by identifying experimental isotopic peaks corresponding to a precursor ion generated from the deprotonated oligonucleotide; determining characteristics of the precursor ion; identifying experimental isotopic peaks corresponding to a fragment ion generated from the precursor ion; determining characteristics of the fragment ion; selecting a candidate fragment for the fragment ion; determining mass shifted isotopic peaks of the candidate fragment based on data that include the characteristics of the precursor ion and the characteristics of the fragment ion; comparing the experimental isotopic peaks corresponding to the fragment ion and the mass shifted isotopic peaks of the candidate fragment; and identifying the fragment ion as the candidate fragment based on the comparing.

### RELATED APPLICATIONS

This application claims priority to U.S. provisional application No. 63/347,946 filed on Jun. 1, 2022, entitled “Systems and Methods for Sequencing Dissociation Spectra of Oligonucleotides Obtained by Negative Electron Activated Dissociation,” which is incorporated herein by reference in its entirety.

### TECHNICAL FIELD

The present disclosure relates generally to mass spectrometry of oligonucleotides, and more specifically to methods and systems for analyzing dissociation spectra of oligonucleotides acquired by electron detachment dissociation and negative electron transfer dissociation to, for example, determine the nucleotide sequence of an oligonucleotide.

### BACKGROUND

Mass spectrometry (MS) is an analytical technique for determining the elemental composition of a substance. Specifically, MS measures a mass-to-charge ratio (m/z) of ions generated from a test substance. MS may be used to identify unknown compounds, to determine isotopic composition of elements in a molecule, to determine the structure of a particular compound by observing its fragmentation (hereinafter also alternatively called dissociation), and to quantify the amount of a particular compound in a sample. Mass spectrometers detect ions and as such, a test sample must be converted to an ionic form during mass analysis. Generally, a mass spectrometer may include an ion source and a mass analyzer. The ion source converts a test sample into gaseous ions and the mass analyzer obtains mass spectra based on their m over z ratios (m/z). In some cases, the mass spectrometer may further include one or more isolation devices installed between the ion source and the mass analyzer; or additionally one or more dissociation device between the isolation device and the mass analyzer.

A mass spectrometer may employ dissociation to cause the fragmentation of large analytes (e.g., oligonucleotides, DNA, RNA, etc.) into smaller fragment ions (e.g., fragments containing individual nucleobases). These smaller fragment ions may then be separated and quantified based on their m/z ratios.

For dissociation, different techniques may be used. For example, a common dissociation technique for biomolecule analysis is the collision induced dissociation (CID). Further, some other techniques that utilize radical driven dissociation methods may be used to obtain information that complement information derived from CID. Such other techniques may include electron capture dissociation (ECD), electron transfer dissociation (ETD), electron detachment dissociation (EDD), or photo dissociation using UV laser (UVPD).

The process of the electron based dissociations may, however, generate a cascade of charge reduced precursor species. For example, after a precursor loses one electron, the resultant precursor ion may be dissociated or may, before being dissociated, lose one or more electrons. Therefore, multiple offsprings of the precursors with different charges may coexist and appear in the spectrometry results. Moreover, these ions and produced fragments from different charges may appear in mass spectra at different locations in the m/z scale. Such complications in the case of electron driven dissociation may, therefore, pose challenges in the interpretation of a spectrum.

### SUMMARY

In some embodiments, the techniques described herein relate to a method of analyzing mass spectra of a deprotonated oligonucleotide, the method including: identifying experimental isotopic peaks corresponding to a precursor ion generated from the deprotonated oligonucleotide; determining one or more characteristics of the precursor ion; identifying experimental isotopic peaks corresponding to a fragment ion generated from the precursor ion; determining one or more characteristics of the fragment ion; selecting a candidate fragment for the fragment ion; determining mass shifted isotopic peaks of the candidate fragment based on data that include the one or more characteristics of the precursor ion and the one or more characteristics of the fragment ion; comparing the experimental isotopic peaks corresponding to the fragment ion and the mass shifted isotopic peaks of the candidate fragment; and identifying the fragment ion as the candidate fragment based on the comparing.

In some embodiments, the techniques described herein relate to a method, wherein the fragment ion is generated from the precursor ion as a result of a negative electron activated dissociation.

In some embodiments, the techniques described herein relate to a method, wherein the one or more characteristics of the precursor ion include a charge of the precursor ion and a mass of the precursor ion.

In some embodiments, the techniques described herein relate to a method, further including determining a length of the precursor ion based on the mass of the precursor ion.

In some embodiments, the techniques described herein relate to a method, wherein the one or more characteristics of the fragment ion include a charge of the fragment ion and a mass of the fragment ion.

In some embodiments, the techniques described herein relate to a method, further including determining a dissociation site corresponding to the fragment ion based on the mass of the fragment ion.

In some embodiments, the techniques described herein relate to a method, wherein determining the mass shifted isotopic peaks of the candidate fragment includes estimating a mass shift equal to integer part of [z−(|Z|−1)i/N], in which z is a charge of the fragment ion, i a number of nucleotides in the fragment ion, |Z| is an absolute value of a charge of the precursor ion, and N is a number of nucleotides in the precursor ion.

In some embodiments, the techniques described herein relate to a method, wherein the deprotonated oligonucleotide is multiply deprotonated.

In some embodiments, the techniques described herein relate to a method, wherein the fragment ion is generated from the precursor ion after removal of two or more electrons from the precursor ion.

In some embodiments, the techniques described herein relate to a method, wherein the fragment ion is generated from the precursor ion as a result of a negative electron activated dissociation after the removal of the two or more electrons from the precursor ion.

In some embodiments, the techniques described herein relate to a system for analyzing mass spectra of a deprotonated oligonucleotide, the system including: a mass spectrometer configured to collect mass spectrometry data of the deprotonated oligonucleotide; and an analyzer module configured to receive the mass spectrometry data and to analyze the mass spectrometry data, wherein analyzing the mass spectrometry data includes: identifying experimental isotopic peaks corresponding to a precursor ion generated from the deprotonated oligonucleotide; determining one or more characteristics of the precursor ion; identifying experimental isotopic peaks corresponding to a fragment ion generated from the precursor ion; determining one or more characteristics of the fragment ion; selecting a candidate fragment for the fragment ion; determining mass shifted isotopic peaks of the candidate fragment based on data that include the one or more characteristics of the precursor ion and the one or more characteristics of the fragment ion; comparing the experimental isotopic peaks corresponding to the fragment ion and the mass shifted isotopic peaks of the candidate fragment; and identifying the fragment ion as the candidate fragment based on the comparing.

In some embodiments, the techniques described herein relate to a system, wherein the fragment ion is generated from the precursor ion as a result of a negative electron activated dissociation.

In some embodiments, the techniques described herein relate to a system, wherein the one or more characteristics of the precursor ion include a charge of the precursor ion and a mass of the precursor ion.

In some embodiments, the techniques described herein relate to a system, wherein analyzing the mass spectrometry data further includes determining a length of the precursor ion based on the mass of the precursor ion.

In some embodiments, the techniques described herein relate to a system, wherein the one or more characteristics of the fragment ion include a charge of the fragment ion and a mass of the fragment ion.

In some embodiments, the techniques described herein relate to a system, wherein analyzing the mass spectrometry data further includes determining a dissociation site corresponding to the fragment ion based on the mass of the fragment ion.

In some embodiments, the techniques described herein relate to a system, wherein determining the mass shifted isotopic peaks of the candidate fragment includes estimating a mass shift equal to integer part of [z−(|Z|−1)i/N], in which z is a charge of the fragment ion, i a number of nucleotides in the fragment ion, |Z| is an absolute value of a charge of the precursor ion, and N is a number of nucleotides in the precursor ion.

In some embodiments, the techniques described herein relate to a system, wherein the deprotonated oligonucleotide is multiply deprotonated.

In some embodiments, the techniques described herein relate to a system, wherein the fragment ion is generated from the precursor ion after removal of two or more electrons from the precursor ion.

In some embodiments, the techniques described herein relate to a system, wherein the fragment ion is generated from the precursor ion as a result of a negative electron activated dissociation after the removal of the two or more electrons from the precursor ion.

Further understanding of various embodiments of the embodiments may be obtained by reference to the following detailed description in conjunction with the associated drawings, which are described briefly below.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in this specification and constitute a part of it, illustrate several embodiments consistent with the disclosure. Together with the description, the drawings serve to explain the principles of the disclosure.

In the drawings:

FIG. 1 shows a diagram illustrating different dissociation sites and naming conventions for the fragments of an exemplary oligonucleotide as utilized in some embodiments.

FIG. 2 includes a diagram 200, which illustrates the processes of deprotonation and fragmentation resulting from collision induced dissociation (CID) of a precursor species 210, the corresponding naming conventions, and the structure of the resulting ions and fragments as utilized in some embodiments.

FIG. 3 shows a panel 300 including 5 mass spectrum sections 310-350, each indicating fragment peaks produced by EDD from a DNA, according to some embodiments.

FIGS. 4A-4C illustrate examples of dissociations according to different embodiments.

FIG. 5 shows a block diagram of a mass spectrometry system 500 that collects the experimental data and analyzes those data according to some embodiments.

FIG. 6 shows a flow chart for a method 600 to determine mass shift for a CRS detected by a mass spectrometry system according to some embodiments.

FIG. 7 shows a flow chart for a method 700 to determine mass shift for fragments produced by nExD, according to some embodiments.

FIG. 8 shows a 13C peak profile panel 800, which corresponds to panel 300 after the analyzer module determine the mass shift for each isotope of the fragment that is identified for each section in panel 300, according to some embodiments.

FIG. 9 schematically depicts an example of an implementation of a module 900 according to some embodiments.

### DETAILED DESCRIPTION

It will be appreciated that for clarity, the following discussion will explicate various aspects of embodiments of the present disclosure, while omitting certain specific details wherever convenient or appropriate to do so. For example, discussion of like or analogous features in alternative embodiments may be abbreviated. For brevity, well-known ideas or concepts may also not be discussed in an any great detail. One of ordinary skill will recognize that some embodiments of the present disclosure may not require certain aspects of the specifically described details in every implementation, which are set forth herein only to provide a thorough understanding of the embodiments. Similarly it will be apparent that the described embodiments may be susceptible to alteration or variation according to common general knowledge without departing from the scope of the disclosure. The following detailed description of embodiments is not to be regarded as limiting the scope of the applicant's teachings in any manner.

Various embodiments provide systems and methods to identify different fragments or species that may result from dissociations at different locations with different numbers or locations for their ionization's.

In various embodiments, the techniques described herein relate to a method of analyzing mass spectra of a deprotonated oligonucleotide produced by electron detachment dissociation (EDD), negative electron transfer dissociations (negative ETD), electron photodetachment dissociation (EPD), and other dissociation methods induced by electron removal.

This disclosure generally defines electron removed species (ERS) as one or more electron removed fragment species or charge reduced precursor species (CRS), and further defines nExD (negative electron activated dissociation) as a general term for dissociations that include EDD, negative ETD, EPD, or other dissociations that may be induced by electron removal.

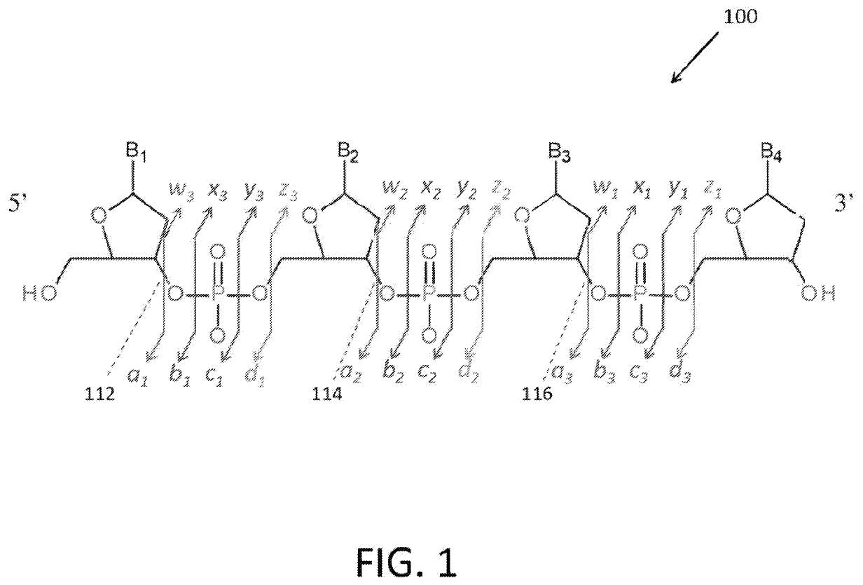

FIG. 1 shows a diagram illustrating different dissociation sites and naming conventions for the fragments of an exemplary oligonucleotide precursor species 100 as utilized in some embodiments. In particular, FIG. 1 illustrates a common nomenclature for the ions produced at dissociation sites along the phosphodiester chain of oligonucleotides species.

More specifically, precursor 100 includes two ends marked by a terminal 3′ (the right hand side) and a terminal 5′ (the left hand side). Also, oligonucleotide precursor 100 includes four nucleotide with bases B1-B4. Further, FIG. 1 illustrates different three dissociation sites 112, 114, and 116, at which precursor 100 may be fragmented by nExD and the naming convention for the corresponding fragments. For example, if precursor 100 is fragmented at dissociation site 112, the resulting left side fragment is named an a₁ fragment, because it includes one base B1 corresponding to terminal 5′ of precursor 100. Moreover, the resulting right side fragment is named a w₃ fragment because it includes 3 bases B2-B4 corresponding to terminal 3′ of precursor 100. In a similar manner, if precursor 100 is fragmented at dissociation site 114, the left and right fragments are respectively named a₂ and w₂; and if precursor 100 is fragmented at dissociation site 116, the left and right fragments are respectively named a₃ and w₁. FIG. 1 also illustrates the naming conventions for fragments generated if the dissociation occurs at other sites. More precisely, the a fragments produced by nExD are radical species denoted as a′, in which the dot in the superscript indicates the missing electron that generates the radical.

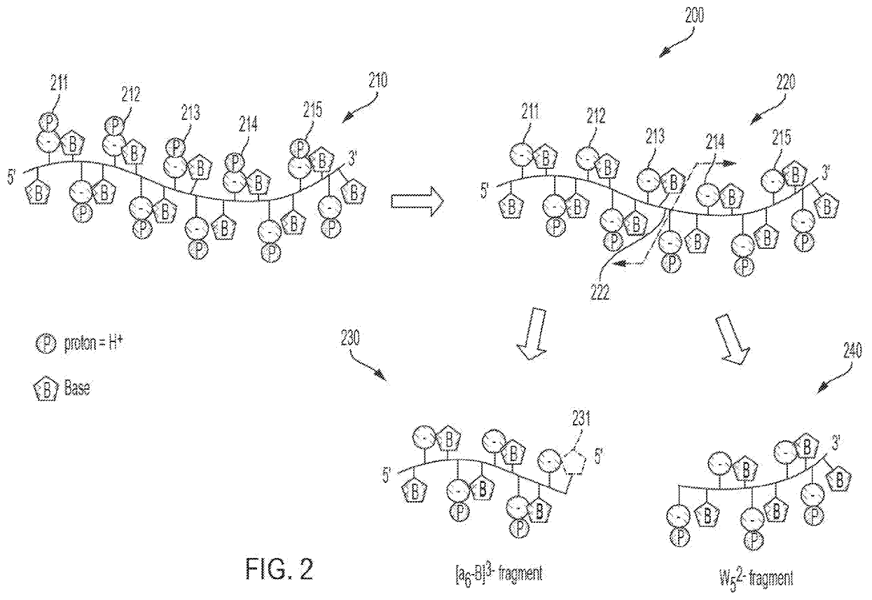

FIG. 2 includes a diagram 200, which illustrates the processes of deprotonation and fragmentation resulting from collision induced dissociation (CID) of a precursor species 210, the corresponding naming conventions, and the structure of the resulting ions and fragments as utilized in some embodiments. More specifically, precursor 210 includes 11 bases, labeled B. Moreover, precursor 210 includes 10 phosphate groups each illustrated by a filled circle labeled with a minus sign attached to another filled circle labeled P, standing for a proton that is equivalent to a hydrogen ion. Further, similar to the two ends of precursor 100 in FIG. 1, the right and left ends of precursor 210 are respectively labeled 3′ and 5′. Precursor 210 is a neutral molecule and may be represented by M.

Diagram 200 further includes a precursor ion 220. Precursor ion 220 may be generated from precursor 210 by deprotonation. Therefore, in some embodiments, such a precursor ion may also be called a deprotonated oligonucleotide precursor species, or more concisely a deprotonated oligonucleotide.

More particularly, precursor ion 220 may be generated from precursor 210 by electro spray ionization in negative mode, which may remove protons from the precursor ions. In diagram 200, five protons located at positions 211-215 are removed from precursor 210 to generate precursor ion 220. As a result, precursor ion 220 has an overall charge of negative 5(Z=5−). Therefore, in some embodiments, precursor ion 220 is represented by the notation or symbol [M−5H]⁵⁻, in which the [M−5H] part indicates that precursor ion 220 is generated from M (precursor 210) by subtracting (removing) five H (hydrogen ions, that is, protons), and the superscript 5− indicates its overall charge of negative 5(Z=5−). The details shown in diagram 200 and subsequent structures, correspond to ionizations or fragmentations of DNA type oligonucleotides. Similar concepts and methods may apply to other types of oligonucleotides, such as RNAs with straightforward modifications.

Diagram 200 further illustrates a fragmentation of DNA via CID that may happen for precursor ion 220 according to some embodiments. More specifically, in the example of diagram 200 precursor ion 220 may be fragmented at dissociation site 222. The fragmentation may result from processes such as molecular vibration or thermal excitation. Such a fragmentation may generate two fragment ions 230 and 240, as shown in diagram 200. Fragment ion 230 is noted as [a₆−B]³⁻ and fragment ion 240 is noted as

$${w}_{5}^{2 -} .$$

In the [a₆−B]³⁻ notation for fragment ion 230, the letter indicates its type and the subscript 6, indicates its dissociated sites or the length (having 6 bases) as described in FIG. 1. In the case of CID applied to DNA, such as shown in diagram 200, fragment ion 230 also loses the 6^{th} base located at location 231. This base loss is often indicated by “−B”, as seen here. Moreover, the charge superscript 3− indicates that fragment ion 230 has a charge of negative 3(z=3−). By convention, for such fragments, the charge superscript also indicates the number of protons missing from the fragment. In other words, the superscript 3− in the notation [a₆−B]³⁻ indicates that fragment ion 230 is missing 3 protons, which in this case correspond the protons that are missing from positions 211-213. Similarly, the w₅²⁻ notation for fragment ion 240 indicates that fragment ion 240 is of type w, includes 5 bases, and is missing two protons (in this case from positions 214 and 215) as a result of which it has a charge of negative 2.

Various embodiments use mass spectrometry results to discover fragments produced by radical induced dissociations. In some embodiments, radical induced dissociations or nExD that are applicable to oligonucleotides include electron detachment dissociation (EDD), negative electron transfer dissociation (ETD), and electron photodetachment dissociation (EPD). Each of these dissociations, may be triggered by removal of one or more electrons. In EDD, an electron in the precursor ions is “detached” by irradiation of an energetic electron beam, typically with an electron kinetic energy higher than 10 eV. In negative ETD, positive reagent ions are often trapped simultaneously with the precursor negative ions in an ion trap to induce electron transfer from the precursor ions to the reagent ions. In EPD, an UV laser beam is irradiated to the precursor ions to induce photoelectric electron detachment. In some embodiments, the term “electron removal” may be used summarily to represent the technique for removal of the electron, including electron transfer electron detachment, electron photodetachment, etc.

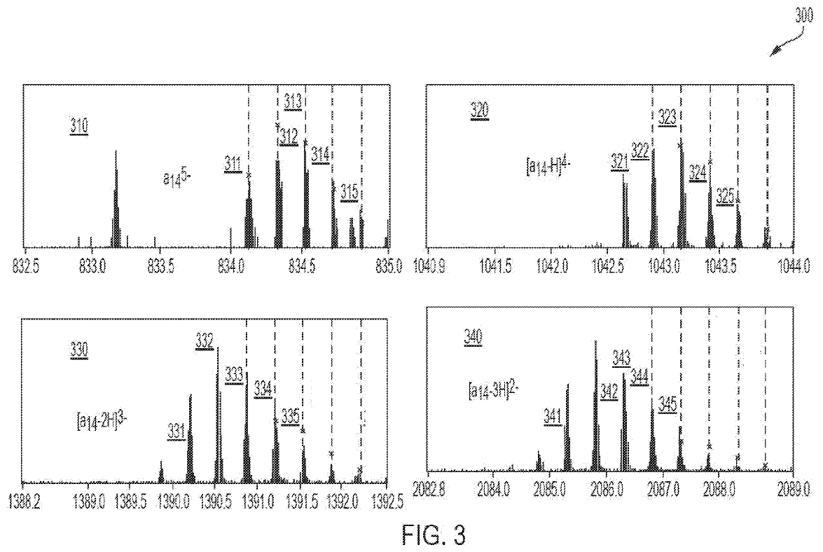

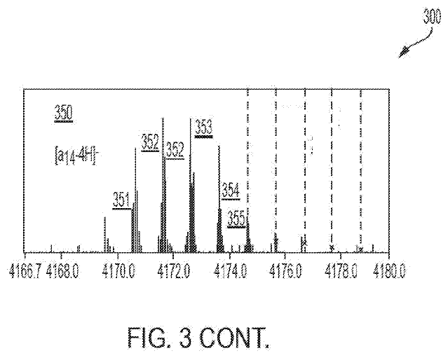

FIG. 3 shows a panel 300 including 5 mass spectrum sections 310-350, each indicating an isotopic peak profile (explained below) for fragment ions that are generated from a DNA by EDD, according to some embodiments. More specifically, sections 310, 320, 330, 340, and 350 show the isotropic profiles of a₁₄ fragments with z=5−, 4−, 3−, 2−, and 1−, respectively. In general, an isotopic peak profile (here alternatively called a ¹³C peak profile) may correspond to a group of peaks (in this case five peaks, as seen in each of sections 310-350) that correspond to different number of carbon 13 isotopes in the same fragment. An isotopic peak profile may be recognized as a set of peaks that are clustered together compared to the other peaks, are spread at equal distances (explained below), and possibly form a Poisson distribution that depends on the average number of ¹³C isotopes in the specific fragment species.

In particular, section 310 shows a spectrum that includes five peaks 311-315 corresponding to five different numbers of contained ¹³C isotopes of a-type fragment according to an embodiment. Each section displays the intensity of the spectrum as a function of a mass to charge ratio (m/z), the values of which are indicated on the horizontal axis. For the m/z values on the horizontal axis, the mass m is measured in the atomic mass units (amu) and the charge z is measured in the elementary charge units (e), and therefore the values are in amu/e units. More specifically, peaks 311-315 correspond to five isotopes of a fragment

$${a}_{14}^{5 -} ,$$

that is, an romzeu naginem or type a, which includes 14 bases and is missing 5 protons, as a result of which it has a charge of negative 5(Z=−5). The five isotopes defer by the number of carbon 13 (¹³C) isotopes of carbon. More specifically, peaks 311-315 respectively correspond to isotopes that include 0-4 carbon 13 isotopes. The first peak, peak 311, is located at m/z value around 834.1 and the last peak, peak 315, is located at m/z value around 834.9. Therefore, the distance between consecutive peaks corresponds to the mass difference between ¹³C and ¹²C (approximately 1.00335 [amu]) divided by the charge of the fragment (in this case 5). As explained below, this relationship may be used to determine, from the distance between the consecutive peaks, the charge of the detected species, such as, a precursor ion, a CRS, or a fragment ion.

In some embodiments, one or more electrons are removed from the precursor ions by multiple electron detachment or electron transfer process without dissociation. The removal of one or more electrons lowers the charge of the precursor ion, but does not change the degree of deprotonation. By way of example and in some embodiments, such as those utilizing EDD, a precursor ion such as [M−7H]⁷⁻ may undergo one or more of the following chain of electron detachment reactions, during which at each stage a new precursor ion (or charge reduced species) is generated, which has the same mass but one less electron compared to the starting precursor ion and therefore an overall charge that differs by a value of −1:

$${[ M - 7 H ]}^{7 -} + e - {▯ [ M - 7 H ]}^{6 -} + 2 e -$$

$${[ M - 7 H ]}^{6} + e - {▯ [ M - 7 H ]}^{5 -} + 2 e -$$

$${[ M - 7 H ]}^{5 -} + e - {▯ [ M - 7 H ]}^{4 -} + 2 e -$$

$${[ M - 7 H ]}^{4 -} + e - {▯ [ M - 7 H ]}^{3 -} + 2 e -$$

$${[ M - 7 H ]}^{3 -} + e - {▯ [ M - 7 H ]}^{2 -} + 2 e -$$

$${[ M - 7 H ]}^{2 -} + e - {▯ [ M - 7 H ]}^{-} + 2 e -$$

For example, in the first electron detachment, [M−7H]⁷⁻ loses one electron and turns into the precursor ion [M−7H]⁶⁻. In some embodiments this second generation precursor ion [M−7H]⁶⁻ may be detected by a mass spectrometer. Alternatively, and in some embodiments, this second generation precursor ion [M−7H]⁶⁻ may itself be subjected to an electron detachment in accordance to the second reaction below, thus generating a precursor ion [M−7H]⁵⁻, and so on.

Alternatively, in some embodiments utilizing negative ETD, a precursor ion such as [M−7H]⁷⁻ may undergo one or more of the following chain of electron transfer reactions using nitrogen ions as the reagent. Similar to the chain of electron detachment reactions described above, here also at each stage a new precursor ion (or charge reduced species) is generated, which has the same mass but one less electron compared to the starting precursor ion and therefore an overall charge that differs by a value of −1.

$${[ M - 7 H ]}^{7 -} + {N}_{2}^{-} {▯ [ M - 7 H ]}^{6 -} + {N}_{2}$$

$${[ M - 7 H ]}^{6} + {N}_{2}^{-} {▯ [ M - 7 H ]}^{5 -} + {N}_{2}$$

$${[ M - 7 H ]}^{5 -} + {N}_{2}^{-} {▯ [ M - 7 H ]}^{4 -} + {N}_{2}$$

$${[ M - 7 H ]}^{4 -} + {N}_{2}^{-} {▯ [ M - 7 H ]}^{3 -} + {N}_{2}$$

$${[ M - 7 H ]}^{3 -} + {N}_{2}^{-} {▯ [ M - 7 H ]}^{2 -} + {N}_{2}$$

$${[ M - 7 H ]}^{2 -} + {N}_{2}^{-} {▯ [ M - 7 H ]}^{-} + {N}_{2}$$

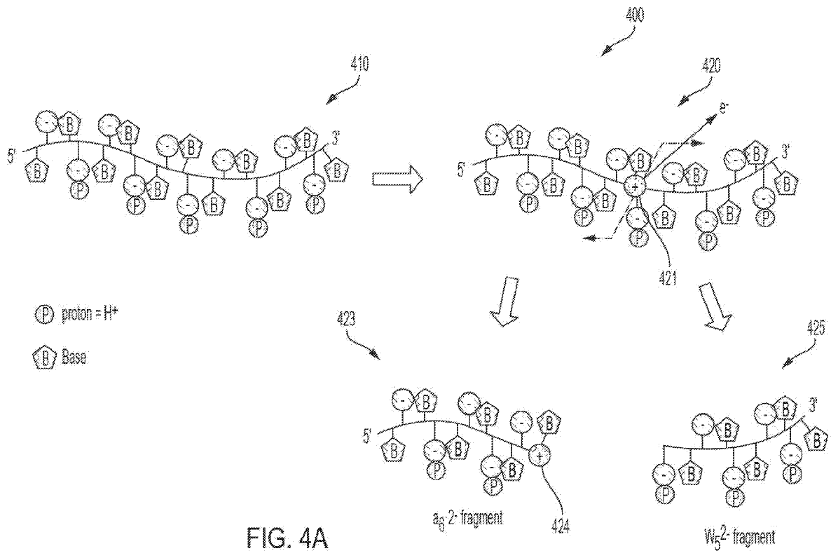

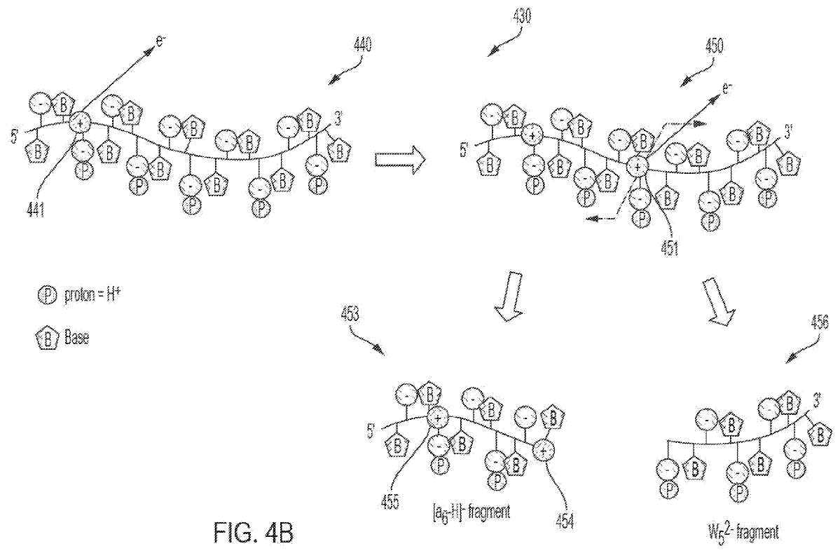

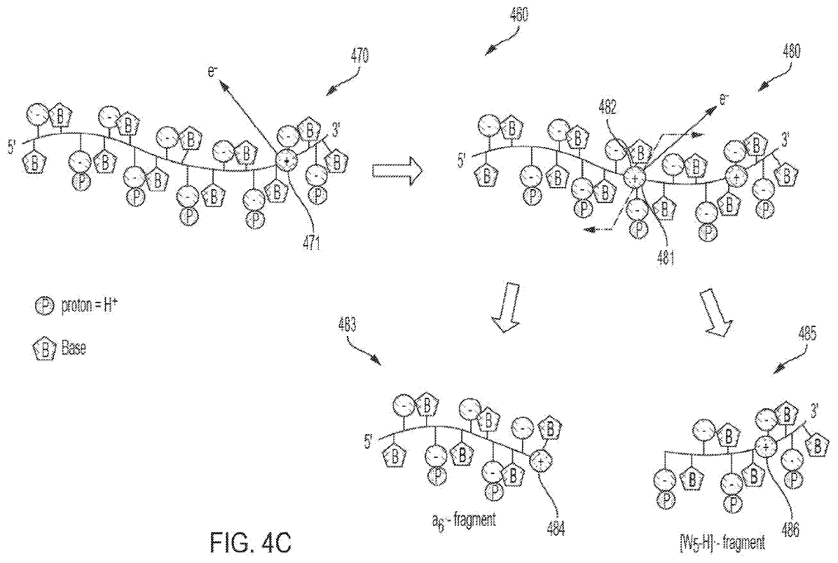

In some embodiments, after a precursor ion loses one or more electrons, it may be dissociated and generate one or more fragments that are also missing those electrons. FIGS. 4A-4C illustrate examples of such dissociations according to different embodiments. More specifically, FIG. 4A includes a diagram 400 that illustrates a precursor ion that loses an electron by one of the techniques of electron removal and then, due to the electron loss, undergoes an nExD. FIG. 4B and FIG. 4C, on the other hand, respectively include diagrams 430 and 460 that illustrate a precursor ion that first loses an electron without dissociation, and then loses a second electron that triggers an nExD.

More specifically, in diagram 400 of FIG. 4A, precursor ion 410 loses an electron from position 421. As a result of the removal of the electron, precursor ion 410 transforms into a CRS 420 (charge reduced species). CRS 420 is also missing 5 protons at the same positions as precursor ion 410. Therefore, its overall charge is negative 4 and is displayed with the notation [M−5H]^{·4-}. This notation shows that CRS 420, has an overall charge of negative 4 and has also lost five protons. Balancing the loss of four protons (which adds a charge of negative 5 to the neutral precursor M) and the overall charge of negative 4 indicates that CRS 420 must have also lost one electron compared to the precursor ion 410 that is notated as [M−5H]5−. In FIG. 4A, the position of the electron loss is indicated as an electron hole in the molecular ions (the filled circle with a plus sign). Similarly, in the notation [M−5H]^{·4-}, the existence of an unpaired electron is indicated by a dot before the indication of the charge (4−) in the superscript.

The nExD, induced after the first electron loss (such as the dissociation at position 421), produces a 5′ terminal fragment 423 and a 3′ terminal fragment 425. In the case of DNA shown in FIG. 4A, the 5′ terminal fragment 423 is a₆^{·2-} and the 3′ terminal fragment 425 is w₅²⁻. The a type fragment ion 423 possesses the unpaired electron or the electron hole 424.

In some embodiments, the CRS itself plays the role of a precursor ion, because the CRS may undergo one or more additional electron removals and generate species that are herein called 2^{nd} reduced species. Those 2^{nd} reduced species may then undergo dissociations such as nExD, and create fragment ions. Diagram 430 in FIG. 4B shows such a process of nExD according to some embodiments. More specifically, diagram 430 shows a CRS 440 that is produced by the 1^{st} electron removal from position 441, but is not dissociated at this time. The position of the electron hole (here at position 441) may be random across the chain of oligonucleotide. CRS 440 may subsequently lose a second electron from position 451, generating 2^{nd} reduced species 450 that includes a second unpaired electron or electron hole at position 451. 2^{nd} reduced species 450 may then undergo a fragmentation at position 451. The position of the second electron hole may also be random across the chain of oligonucleotide.

The fragmentation by the second electron removal creates two fragment ions, fragment ion 453 and fragment ion 456. Fragment ion 456, similar to fragment ion 425, is a

$${w}_{5}^{2 -}$$

fragment. Fragment ion 453, on the other hand, while missing 3 protons similar to fragment ion 423, is different from fragment ion 423 in that fragment ion 453 is also missing two electrons at positions 454 and 455, while fragment ion 423 is missing one electron at position 424. Therefore, while fragment ion 423 has a charge of negative 2, fragment ion 453 has a charge of negative 1. To illustrate this difference with fragment ion 423 (which is displayed as

$${a}_{6}^{2 -} ) ,$$

fragment ion 453 is displayed with the notation [a₆−H]⁻. The notation [a₆−H]⁻ may be interpreted by convention as follows. The combination of the letter a and the subscript 6 indicates a type fragment with 6 bases. Further, the superscript “−” indicates the fragment 423 has an overall charge of negative 1, resulting from removal of one proton from the standard a^{·} fragment, a₆^{·-} that is, missing an overall number of two protons, and also missing one electron.

Next, considering diagram 460 in FIG. 4C, which, similar to FIG. 4B illustrates a process through which a precursor ion first undergoes electron depletion and next an nExD according to some embodiments. Diagram 460 shows that the precursor ion loses an electron through an electron removal from position 471 near 3′ terminus. As a result of the removal of the electron, the precursor ion transforms into a CRS 470 (which is also missing 5 protons at the same positions as the precursor ion) has an overall charge of negative 4. As a result, CRS 470 is displayed with the notation [M−5H]^{·4-}, indicating that CRS 470 has an overall charge of negative 4 that results from losing 5 protons and one electron.

Diagram 460 also shows that CRS 470 may further lose another electron at a position 482 and generate 2^{nd} reduced species 480, which may then undergo an nExD at position 482. The fragmentation creates two fragment ions, fragment ion 483 and fragment ion 485. Fragment ion 483, similar to fragment ion 423. is an

$${a}_{6}^{2 -}$$

fragment, including six bases and having a charge of negative 2. Fragment ion 485, on the other hand, while missing two protons similar to fragment ion 425, is different from fragment ion 425 because fragment ion 485 is also missing an electron at a position 486. Therefore, while fragment ion 425 has a charge of negative 2, fragment ion 485 has instead a charge of negative 1. To illustrate this difference with fragment ion 425 (which is displayed as

$${w}_{5}^{2 -} ) ,$$

fragment ion 485 is displayed with the notation [w₅−H]^{·-}. As described above, the notation [w₅−H]^{·-} indicates fragment ion is of the w type, includes 5 bases, and is missing 2 protons and 1 electron, therefore having charge of negative 1.

The above described phenomenon of electron depletion of a precursor ion in the CRS or electron depletion of a fragment to generate a fragment ion may cause errors in analyzing mass spectrometry results. Such errors may result in false positive or false negative. In some embodiments, nExD products that are produced from the precursor species by the 1^{st} electron detachment or 1^{st} electron transfer may cause a shift in the profile associated with a fragment. In some embodiments, the shift is due to the difference between the expected mass of the fragment and the real mass of the fragment. In some embodiments, this shift is called a mass shift, further detailed below.

Panel 300 of FIG. 3 illustrates examples of possible errors that may arise and corrected according to some embodiments. Section 320, for example, shows 5 peaks 321-325 generated by 0 to 4 ¹³C isotopes of the fragment [a₁₄−H]⁴⁻. As explained earlier, the mass of [a₁₄−H]⁴⁻ is equal to the mass of

$${a}_{14}^{5 -} .$$

Unlike the fragment ion

$${a}_{14}^{5 -} ,$$

which has a charge of negative 5, however, the fragment ion [a₁₄−H]⁴⁻ has a charge of negative 4 (due to the one removed electron). Therefore, because m/z is the value of mass divided by charge, the m/z value for the fragment ion

$${a}_{14}^{5 -}$$

is equal to ⅘^{th} of the m/z value for that fragment ion [a₁₄−H]⁴⁻. This relation is verified by comparing the m/z value for peak 311 (corresponding to the zeroth isotope, i.e., an isotope with no carbon 13 or the monoisotopic peak of the fragment ion

$${a}_{14}^{5 -} ) ,$$

which is approximately 834.1 with the m/z value of peak 321 (corresponding to the zeroth isotope of the fragment ion [a₁₄−H]⁴⁻), which is approximately 1042.6.

Therefore, the isotopic peak profile of the fragment ion [a₁₄H]⁴⁻ is located in the same m/z range as the fragment ion

$${a}_{14}^{4 -} ,$$

because they have the same charge (negative 4 units) and, approximately, the same mass. For example, the zeroth isotope of [a₁₄ −H]⁴⁻ is identical to the zeroth isotope of

$${a}_{14}^{4 -} ,$$

except for missing one additional proton (indicated by “−H” in the brackets). Therefore, peak 321, which is the isotopic peak for the fragment ion [a₁₄−H]⁴⁻, is shifted to the left as compared to the location of the isotopic peak for the fragment ion

$${a}_{14}^{4 -} ,$$

which would be located at the location of peak 322. The magnitude of the shift, which is the distance between peaks 321 and 322, is equal to the differences in the mass (1 amu) divided by the magnitude of the charge (here 4). Noteworthy is that the magnitude of this shift is the same as the magnitude of the shift between consecutive isotopes of the same charge. Therefore the peaks for the first to fourth isotopes of the fragment ion [a₁₄−H]⁴⁻ are also located at the location of the zeroth to the third isotopes of the fragment ion

$${a}_{14}^{4 -} .$$

[a₁₄−H]⁴⁻ can respectively be derived by finding the five peaks in the isotopic peak profile for the fragment ion

$${a}_{14}^{4 -}$$

and shifting them to the left by one shift unit (defined as 1 amu divided by the magnitude of the charge, which is 1 divided by 4=0.25 for this set of peaks). Therefore, because the shift is much smaller than the value of the m/z for each peak, the isotopic peak profile for the fragment ion [a₁₄−H]⁴⁻ may be mistaken for, and miscounted as, an isotopic peak profile for the fragment ion

$${a}_{14}^{4 -} .$$

Sections 330-350 illustrate similar errors caused by mass shifts associate with the fragment a₁₄. More specifically, in section 330, peaks 331-335 respectively correspond to the zeroth-fourth isotopes of [a₁₄−2H]³⁻, the locations of which overlap the locations of the zeroth-fourth isotopes of the fragment ion

$${a}_{14}^{3 -}$$

(which are indicated by the dotted lines, normally located at positions 333-337) after being shifted to the left by two shift units (the shift unit in this case being 1 divided by 3=0.33). Similarly, section 340 shows mass spectrum peaks at locations 341-345, corresponding to the isotopes of [a₁₄−3H]²⁻, which appear and may be miscounted as peaks that correspond to the isotopes of the fragment ion

$${a}_{14}^{2 -}$$

(which are indicated by the dotted lines, normally located at positions 344-348) after being shifted to the left by three shift units (the shift unit in this case being 1 amu divided by 2=0.5). Also, section 350 shows mass spectrum peaks at locations 351-355, corresponding to the isotopes of [a₁₄−4H]⁻, which appear and may be miscounted as peaks that correspond to the isotopes of the fragment ion a₁₄⁻ (which are indicated by the dotted lines, normally located at positions 355-349) after being shifted to the left by 4 shift units (the shift unit in this case being 1).

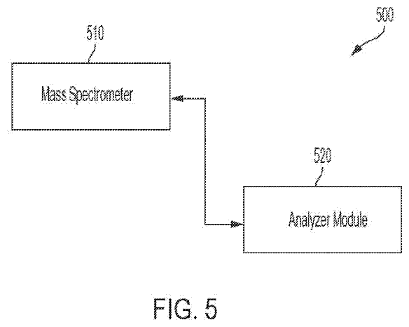

Various embodiments address errors such as those described above, that may be introduced in analyzing mass spectra. FIG. 5 shows a block diagram of a mass spectrometry system 500 that collects the experimental data and analyzes those data according to some embodiments. More specifically, mass spectrometry system 500 includes a mass spectrometer 510 and an analyzer module 520.

Mass spectrometer 510 may collect mass spectrometry data and send them to analyzer module 520. Analyzer module 520, on the other hand, may receive the mass spectrometry data from mass spectrometer 510 and analyze those data to derive histograms or intensities for different fragments present in the spectrum. More specifically, analyzer module 520 may identify experimental isotopic peaks corresponding to isotopic species of a fragment of a deprotonated oligonucleotide, detect from the peaks a CRS that may be generated from the deprotonated oligonucleotide or detect one or more expected fragments that may be generated via nExD of the CRS. Further, analyzer module 520 may determine mass shifts associated with the nExD and eventually identify isotopic species that correspond to the experimental isotopic peaks.

For example, analyzer module 520 may analyze mass spectrometry results such as those in mass spectrum panel 300, and accordingly identify the isotopic peaks such as those shown in sections 320-350 as corresponding to the correct fragments (identified in the description of each section above). The analyzer module may thus correct the above-described overcounts or undercounts.

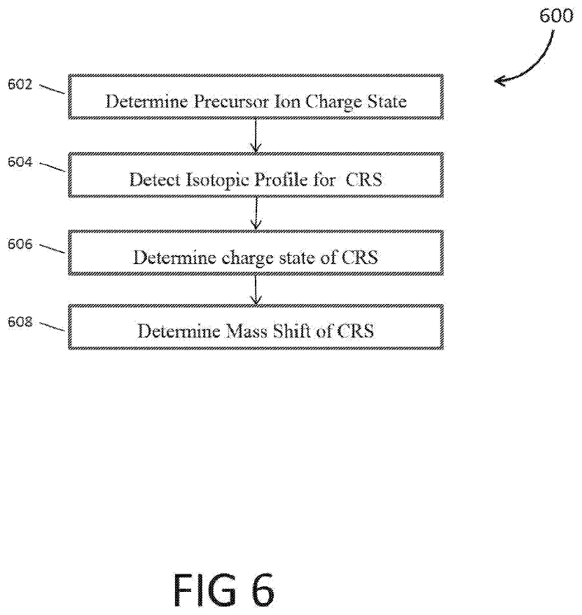

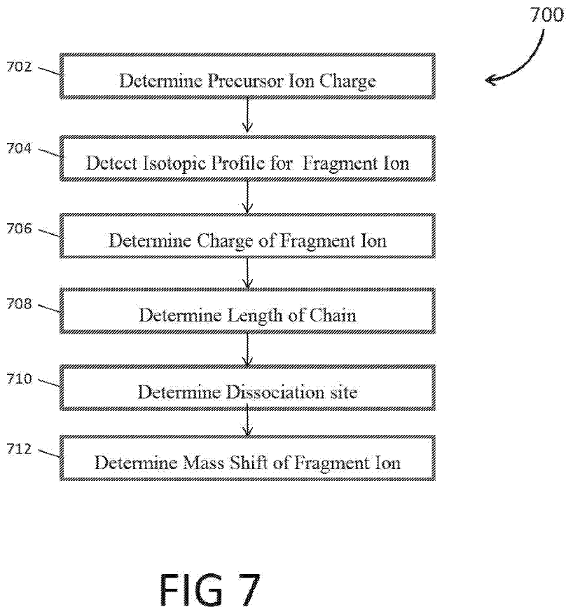

In particular, FIG. 6 shows a flow chart for a method 600 to determine mass shift for a CRS detected by a mass spectrometry system according to some embodiments. Similarly, FIG. 7 shows a flow chart for a method 700 to determine mass shift for fragment ions produced by nExD according to some embodiments. In some embodiments, the ions that are generated after one or more electron removals and detected before an nExD (as is the case for a detected CRS) or after an nExD (as is the case for the fragment ions 453 or 485) may generally be called an electron removed species (ERS).

As a first step, at step 602 of method 600 or step 702 of method 700, the analyzer module may determine a charge (Z) for the precursor ion that corresponds to the ERS. To that end, the analyzer module may first detect a set of peaks that correspond to an isotopic profile for the precursor ion. The analyzer module may identify such a set of peaks based on their pattern, for example, as set of peaks that are clustered together compared to other peaks, or together form the Poisson peak height distributions discussed above.

The analyzer module may then determine Z by using the relation shown in Eq. (1):

$$Z = M {(}_{13 C - 12 C )} / Δ m . \qquad ( 1 )$$

In Eq. (1), M(_{13C-12C}) is the mass difference between the two isotopes of carbon (¹³C and ¹²C) and Δm is the difference between the m/z values of consecutive peaks in the isotopic profile of the precursor ion.

Next, the analyzer module may detect an isotopic profile. For example, at step 604 of method 600 in FIG. 6, the analyzer module detects a set of peaks that correspond to an isotopic profile for a CRS. Similarly, at step 704 of method 700 in FIG. 7, the analyzer module detects one or more sets of peaks that correspond to isotopic profiles for one or more fragment ions, such as the set of five peaks seen in any one of sections 320-350 of panel 300. Next, at step 606 of method 600 or step 706 of method 700, the analyzer module

may determine a charge for the corresponding ERS (z). In some embodiments, the analyzer module may determine z in a manner similar to Z, that is, by using the relation in Eq. (1) after inserting for the value of Am the difference between the m/z values of consecutive peaks in the previously detected isotopic profile of the ERS.

Next, at step 608 of method 600, analyzer module 520 determines a mass shift for the CRS. In some embodiments, analyzer module 520 determines this mass shift by using the relation shown in Eq. (2):

$$Mass shift for CRS = Z - z , \qquad ( 2 )$$

in which the mass shift is measured in shift units for the CRS, as defined above.

In some embodiments, Eq. (2) may be explained as follows. The difference between the charges of the precursor ion and the CRS may correspond to the number of electrons removed from the precursor ion to generate the CRS. Moreover, as explained above in the detailed descriptions of sections 320-350 of panel 300, the number of removed electrons may translate into the number of mass shifts in the corresponding isotopic peak profile.

The remainder of method 700 in FIG. 7, on the other hand, shows the steps that the analyzer module may perform to determine one or more mass shifts for one or more fragment ions according to some embodiments. More specifically, at step 708 the analyzer module determines the number of nucleotides (or length) in the analyte oligonucleotide (N). The analyzer module may determine N by first determining the mass of the precursor ion M using Eq. (3):

$$M = MOZ * Z + {M}_{H} * Z . \qquad ( 3 )$$

In Eq. (3), MOZ is the m/z value for the zeroth isotope peak in the isotopic profile of the precursor ion, Z is the charge of the precursor ion determined earlier, and M_{H} is the mass of a hydrogen atom (or a proton) in atomic mass unit (amu).

The analyzer module may then use the determined M and the known average mass (Mₙ_{ₜ}) of the nucleotides (which are, for example, A, T, C, G in the cases of DNA, and A, U, C, G in the cases of RNA) to estimate N based on Eq. (4):

$$N \approx M / {M}_{n t} . \qquad ( 4 )$$

Next, at step 710, the analyzer module may determine the location of the

dissociation site (i) which results in the creation of the fragment ion from the precursor ion. The analyzer module may determine i by first determining the mass of the fragment ion (m) using Eq. (5):

$$m = moz * z + MH * z . \qquad ( 5 )$$

In Eq. (5), moz is the m/z value for the zeroth isotope peak in the isotopic profile of the fragment ion, and z is its charge determined earlier. The analyzer module may then estimate i using Eq. (6):

$$i \approx m / {M}_{n t} . \qquad ( 6 )$$

In some embodiments, as detailed below, the analyzer module needs to determine the value of the ratio of i over N (i/N). In some embodiments, in which a candidate sequence of the oligonucleotide and the fragment ion is known in advance, the analyzer module may determine the ratio (i/N) based on a first method that uses the exact number of the nucleotides in the candidate sequence as N and the exact number of the nucleotides in the fragment as the value of i. Otherwise, the analyzer module may determine that ratio based on a second method that combines Eqs. (4) and (6) to derive Eq. (7) as an approximation for this ratio:

$$i / N \approx m / M . \qquad ( 7 )$$

The analyzer module may then use the value of the ratio (i/N), determined by one of the first or second methods above, to derive the mass shift for the fragment ions as follows. When a mass shift is not induced, which is the case of 1^{st} nExD caused by just one electron removal, the analyzer module may estimate the charge of the regular (or non-shifted) fragment using the approximation shown in Eq. (8):

$$charge of the regular fragment \approx ( \vert Z \vert - 1 ) * i / N . \qquad ( 8 )$$

Based on Eq. (8), at step 712 the analyzer module may determine a mass shift for the isotopic profile of the fragment ion. In some embodiments, the analyzer module determines this mass shift by using the relation shown in Eq. (9):

$$Mass Shift for fragment ion \approx integer part [ z - ( \vert Z \vert - 1 ) i / N ] \qquad ( 9 )$$

in which the mass shift is measured in shift units for the fragment ion, as defined above.

In some embodiments determining the mass shift for the CRS or the fragments enables the analyzer module to correct the possible shift in the spectrometry results and accordingly provide a corrected ¹³C profile for detected CRS or fragments. Accordingly, the analyzer module will derive more accurate result for the structures or chemical compositions of oligonucleotides.

More specifically, in various embodiments, the analyzer module may first enumerate different possible candidate fragments and different possible candidate fragment ions.

Further, using the above methods or systems, the analyzer module may apply the mass shifts to the profiles of the candidate fragments to determine the locations of the profiles for the candidate fragment ions. Then, the analyzer module may search for spectrum results that match one or more of the predicted profiles for the fragment ions and, if found, identify those profiles as corresponding to the candidate fragment ion and therefore more accurately determine the structure of the precursors.

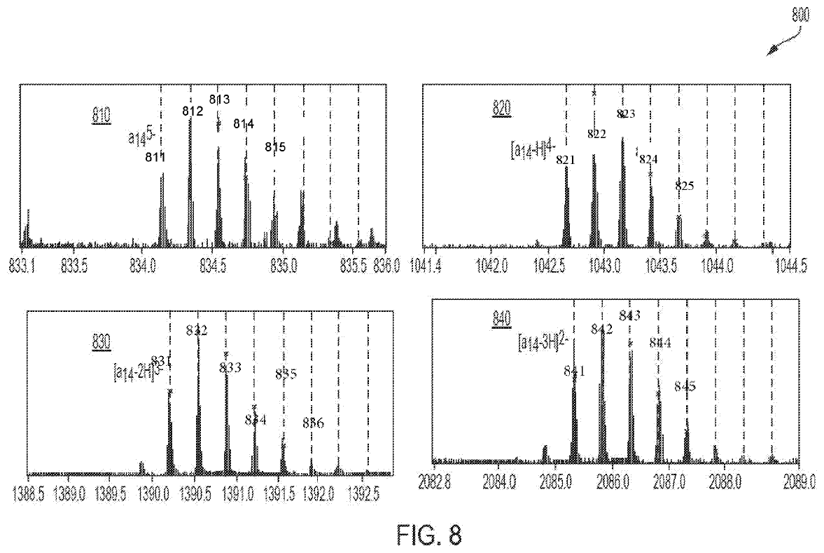

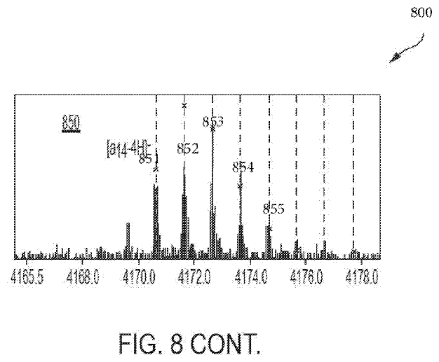

FIG. 8 shows a ¹³C peak profile panel 800, which corresponds to fragment ions mentioned in relation to panel 300, after the analyzer module determines the mass shift for each isotope of the fragment that is identified for each section in panel 300 and applies those mass shifts to the corresponding peak to determine the correct location of the peak. More specifically, sections 820-850 show the predicted locations of the peaks for the profiles of the fragment ions earlier associated with sections 320-350, respectively. In each section, the dotted lines that are labeled by numbers indicate the locations of the corrected peaks for the corresponding isotope. As seen in each of the sections, especially sections 820-850, the corrected locations agree with the experimental data observed in sections 320-350, respectively. More specifically, corrected locations for peaks 821-825 agree with the experimental locations for peaks 321-325, respectively; corrected locations for peaks 831-835 agree with the experimental locations for peaks 331-335, respectively; corrected locations for peaks 841-845 agree with the experimental locations for peaks 341-345, respectively; and corrected locations for peaks 851-855 agree with the experimental locations for peaks 351-355, respectively. Some of the sections include additional data that are considered to possibly correspond to contaminations (seen, for example, in section 810 around 833.1 moz, section 830 slightly below 1390.0 or near 1392.2, section 840 slightly below 2085.0, etc.).

Therefore, the disclosed systems and methods are able to identify and correct the errors that may result from removal of one or more electrons from precursor ions or other species that are detected by the mass spectrometer.

In various embodiments, one or more of disclosed modules may be implemented via one or more computer programs for performing the functionality of the corresponding modules, or via computer processors executing those programs. In some embodiments, one or more of the disclosed modules may be implemented via one or more hardware units executing firmware for performing the functionality of the corresponding modules. In various embodiments, one or more of the disclosed modules may include storage media for storing data used by the module, or software or firmware programs executed by the module. In various embodiments, one or more of the disclosed modules or disclosed storage media may be internal or external to the disclosed systems. In some embodiments, one or more of the disclosed modules or storage media may be implemented via a computing “cloud,” to which the disclosed system connects via a network connection and accordingly uses the external module or storage medium. In some embodiments, the disclosed storage media for storing information may include non-transitory computer-readable media, such as a CD-ROM, a computer storage, e.g., a hard disk, or a flash memory. Further, in various embodiments, one or more of the storage media may be non-transitory computer-readable media that store data or computer programs executed by various modules, or implement various techniques or flow charts disclosed herein.

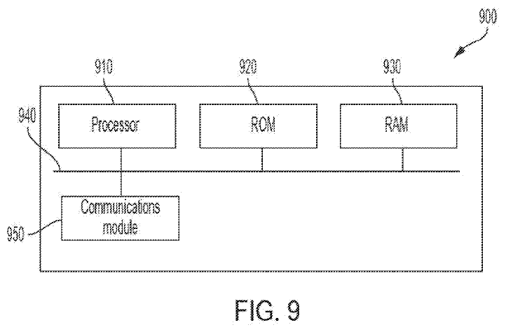

FIG. 9 schematically depicts an example of an implementation of a module 900 according to some embodiments. Module 900 includes a processor 910 (e.g., a microprocessor), at least one permanent memory module (e.g., ROM 920), at least one transient memory module (e.g., RAM) 930, a bus 940, and a communication module 950.

Processor 910, ROM 920, and RAM 930 may be utilized to store and execute instructions performing the function of module 900. Moreover, bus 940 may allow communication between the processor and various other components of the controller. Communication module 950 may be configured to allow sending and receiving signals.

Although some aspects have been described in the context of a system and/or an apparatus, it is clear that these aspects may also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a processor, a microprocessor, a programmable computer, or an electronic circuit. In some embodiments, some one or more of the most important method steps may be executed by such an apparatus.

Those having ordinary skill will appreciate that various changes may be made to the above embodiments without departing from the scope of the invention.

The above detailed description refers to the accompanying drawings. The same or similar reference numbers may have been used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

The foregoing description of the embodiments has been presented for purposes of illustration only. It is not exhaustive and does not limit the embodiments to the precise form disclosed. While several exemplary embodiments and features are described, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. As used herein, the singular forms “a,” “an,” and “the” may include the plural forms unless the context clearly dictates otherwise. Further, the term “coupled” does not exclude the presence of intermediate elements between the coupled items. Also, stating that a feature may exist indicates that the feature may exist in one or more embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or. Moreover, if these terms are used, a subset of a set may include one or more than one, including all, members of the set.

Further, if used in this disclosure, and unless stated or deducted otherwise, a first variable is an increasing function of a second variable if the first variable does not decrease and instead generally increases when the second variable increases. On the other hand, a first variable is a decreasing function of a second variable if the first variable does not increase and instead generally decreases when the second variable increases. In some embodiment, a first variable may be an increasing or a decreasing function of a second variable if, respectively, the first variable is directly or inversely proportional to the second variable.

The disclosed systems, methods, and apparatus are not limited to any specific aspect or feature or combinations thereof, nor do the disclosed systems, methods, and apparatus require that any one or more specific advantages be present or problems be solved. Any theories of operation are to facilitate explanation, but the disclosed systems, methods, and apparatus are not limited to such theories of operation.

Modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments, and may also include other parts not described in the embodiments. Accordingly, the embodiments are not limited to the above-described details, but instead are defined by the appended claims in light of their full scope of equivalents. Further, the present disclosure is directed toward all novel and non-obvious features and aspects of the various disclosed embodiments, alone and in various combinations and sub-combinations with one another.

While the present disclosure has been particularly described in conjunction with specific embodiments, many alternatives, modifications, and variations will be apparent in light of the foregoing description. It is therefore contemplated that the appended claims will embrace any such alternatives, modifications, and variations as falling within the true spirit and scope of the present disclosure.

What is claimed is:

1. A method of analyzing mass spectra of a deprotonated oligonucleotide, the method comprising:
identifying experimental isotopic peaks corresponding to a precursor ion generated from the deprotonated oligonucleotide;
determining one or more characteristics of the precursor ion;
identifying experimental isotopic peaks corresponding to a fragment ion generated from the precursor ion;
determining one or more characteristics of the fragment ion;
selecting a candidate fragment for the fragment ion;
determining mass shifted isotopic peaks of the candidate fragment based on data that include the one or more characteristics of the precursor ion and the one or more characteristics of the fragment ion;
comparing the experimental isotopic peaks corresponding to the fragment ion and the mass shifted isotopic peaks of the candidate fragment; and
identifying the fragment ion as the candidate fragment based on the comparing.

2. The method of claim 1, wherein the fragment ion is generated from the precursor ion as a result of a negative electron activated dissociation.

3. The method of claim 1, wherein the one or more characteristics of the precursor ion include a charge of the precursor ion and a mass of the precursor ion.

4. The method of claim 3, further comprising determining a length of the precursor ion based on the mass of the precursor ion.

5. The method of claim 1, wherein the one or more characteristics of the fragment ion include a charge of the fragment ion and a mass of the fragment ion.

6. The method of claim 5, further comprising determining a dissociation site corresponding to the fragment ion based on the mass of the fragment ion.

7. The method of claim 1, wherein determining the mass shifted isotopic peaks of the candidate fragment comprises estimating a mass shift equal to integer part of [z−(|Z|−1)i/N], in which z is a charge of the fragment ion, i a number of nucleotides in the fragment ion, |Z| is an absolute value of a charge of the precursor ion, and N is a number of nucleotides in the precursor ion.

8. The method of claim 1, wherein the deprotonated oligonucleotide is multiply deprotonated.

9. The method of claim 1, wherein the fragment ion is generated from the precursor ion after removal of two or more electrons from the precursor ion.

10. The method of claim 9, wherein the fragment ion is generated from the precursor ion as a result of a negative electron activated dissociation after the removal of the two or more electrons from the precursor ion.

11. A system for analyzing mass spectra of a deprotonated oligonucleotide, the system comprising:
a mass spectrometer configured to collect mass spectrometry data of the deprotonated oligonucleotide; and
an analyzer module configured to receive the mass spectrometry data and to analyze the mass spectrometry data, wherein analyzing the mass spectrometry data comprises:
identifying experimental isotopic peaks corresponding to a precursor ion generated from the deprotonated oligonucleotide;
determining one or more characteristics of the precursor ion;
identifying experimental isotopic peaks corresponding to a fragment ion generated from the precursor ion;
determining one or more characteristics of the fragment ion;
selecting a candidate fragment for the fragment ion;
determining mass shifted isotopic peaks of the candidate fragment based on data that include the one or more characteristics of the precursor ion and the one or more characteristics of the fragment ion;
comparing the experimental isotopic peaks corresponding to the fragment ion and the mass shifted isotopic peaks of the candidate fragment; and
identifying the fragment ion as the candidate fragment based on the comparing.

12. The system of claim 11, wherein the fragment ion is generated from the precursor ion as a result of a negative electron activated dissociation.

13. The system of claim 11, wherein the one or more characteristics of the precursor ion include a charge of the precursor ion and a mass of the precursor ion.

14. The system of claim 13, wherein analyzing the mass spectrometry data further comprises determining a length of the precursor ion based on the mass of the precursor ion.

15. The system of claim 11, wherein the one or more characteristics of the fragment ion include a charge of the fragment ion and a mass of the fragment ion.

16. The system of claim 15, wherein analyzing the mass spectrometry data further comprises determining a dissociation site corresponding to the fragment ion based on the mass of the fragment ion.

17. The system of claim 11, wherein determining the mass shifted isotopic peaks of the candidate fragment comprises estimating a mass shift equal to integer part of [z−(|Z|−1)i/N], in which z is a charge of the fragment ion, i a number of nucleotides in the fragment ion, |Z| is an absolute value of a charge of the precursor ion, and N is a number of nucleotides in the precursor ion.

18. The system of claim 11, wherein the deprotonated oligonucleotide is multiply deprotonated.

19. The system of claim 11, wherein the fragment ion is generated from the precursor ion after removal of two or more electrons from the precursor ion.

20. The system of claim 19, wherein the fragment ion is generated from the precursor ion as a result of a negative electron activated dissociation after the removal of the two or more electrons from the precursor ion.
